(19) United States
(12) Patent Application Publication
(10) Pub. No.: US 2003/0219465 A1
(43) Pub. Date: Nov. 27, 2003

(54) Composition for delivery of dithranol

(76) Inventors: Suresh Kumar Gidwani, Mumbai (IN); Purushottam Sharshikant Singnurkar, Jalgaon (IN)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 10/371,295

(22) Filed: Feb. 21, 2003

Related U.S. Application Data
(60) Provisional application No. 60/382,997, filed on May 23, 2002.

(51) Int. Cl. A61K 7/075; A61K 7/08; A61K 31/355

(52) U.S. Cl. 424/401; 424/70.21; 514/458

(57) ABSTRACT

Mixed vesicular system containing a nanoemulsion with bilayer nanovesicles mimicking noisome and liposome for the topical delivery of dithranol alone or together with salicylic acid to treat psoriasis, eczemas, dermatophytoses, alopecia areata and other dermatological diseases and methods for producing said system.

### BACKGROUND OF THE INVENTION

Dithranol (1,8,9-trihydroxyanthracene) is used in the topical treatment of psoriasis, eczemas, dermatophytoses, alopecia areata and other dermatological diseases. It suffers however from some serious disadvantages.

It is highly irritant to skin not affected by psoriasis and must therefore be applied with considerable care and often limits the time of exposure of such preparations to the skin. Currently in clinical practice the preparations are to be applied on the skin for short period of and to be removed after 30 minutes of application otherwise it becomes very difficult to tolerate the skin irritation.

It is easily oxidized to brown or black products and has a powerful staining on clothing & normal skin. Commercially in European countries it is known that, dithranol is mixed with ointment base and compounded by the pharmacist in the pharmacy and given to the patient for immediate use. Also British Pharmacopoeia 2001, volume II, page 2035 states that Dithranol Cream should be stored at a temperature not exceeding 25° C.

It is very difficult to deliver dithranol in soluble form in the concentration ranging above 0.15% and it is the soluble form of the dithranol, which is more effective than dispersed or suspended dithranol.

There is a need to have the dithranol compositions, which will be free of above undesirable effects, and those can be applied to the skin for a prolonged period of time to achieve better therapeutic effect in the treatment of psoriasis. Also such preparations should be stable on prolonged storage.

There have been a number of references in the literature to compositions containing dithranol. Such as

Ointments (petroleum base).

Creams with either o/w or w/o emulsion.

Liposomes & Niosomes formulations (Commercially not available).

Solid lipid nanoparticles (Commercially not available).

However they have their own limitations one of the other with respect to stability, efficiency, irritation potential & staining caused by dithranol. One of the major limitation is the low level of dithranol in soluble form and often requires dispersion of dithranol in powder form to achieve dithranol concentration above 0.75% in the product.

Laugier; Jean Pierre et al. in U.S. Pat. No. 5358716 described oil in water emulsion containing dithranol and nonionic vesicles prepared for nonionic amphilic lipids, in order to prevent the oxidation of the dithranol. Vesicles are dispersed in the aqueous phase of the emulsion. However, dithranol is not encapsulated in the vesicles and the o/w emulsion compositions with dithranol content greater than 0.15% are present as micronised dithranol in dispersed form and not in solution form. It is not possible to incorporate dithranol in solution form above 0.15% concentration in such preparations due to limitation of dithranol solubility in oil and lipid phase.

Shyamal, C et al., Journal of Pharmaceutical Science Vol. 85, No. 10, October 1996, 1082-1084; described the topical delivery of Erythromycin from Liposomal emulsion. A nonionic liposomal formulation containing glyceryl dilaurate, cholesterol, polyoxyethylene-10-stearyl ether at weight ratio of 57:15:28 prepared by melting the lipid phase containing Erythromycin base at 60° C. followed by hydration with buffer solution. This liposomal dispersion was mixed with mineral oil or isopropyl palmitate and sonicated to yield mixture of emulsion with liposomes. They showed maximum stability for 2 weeks for such formulations. It is known in the art that any active ingredient/drug present in solution form when applied topically, diffuses more rapidly through the skin as compared to dispersed solid particular drug. It can very well be demonstrated by universal Fick's law of diffusion. It was an object of the invention to overcome the above-mentioned limitations and to formulate efficient and stable delivery system containing dithranol alone or in combination with salicylic acid and coal tar extract for effective and rapid control of psoriasis.

### SUMMARY OF INVENTION

The present invention relates to a mixed vesicular system for the topical delivery of dithranol alone or in combination with salicylic acid which mimics noisomes and liposomes. This system, if desired, can also contain vacuum dried coal tar extract. The mixed vesicular system is composed of a nanoemulsion with bilayer nanovesicles, wherein dithranol and, if present, salicylic acid are entrapped in the vesicles. This is achieved through the use in this system of a nonionic oily liquid lipid material capable of solubilizing dithranol itself or in combination with salicylic acid. The mixed vesicular system has particle size of no greater than 450 nm and can be stored at room temperature for extended periods of time. Furthermore the composition of this invention does not produce irritation or staining and is very effective for the treatment of psoriasis.

It is known that any active ingredient/drug present in solution form diffuses rapidly through the skin as compared to solid dispersed particulate drug. The composition of this invention provides a topical pharmaceutical compositions containing dithranol in soluble form even at higher drug concentrations so as to make it more effective. In this manner, the active ingredients can penetrate into and through the skin so that the active ingredients are provided continuously and in sufficient quantity at the site of action. The topical compositions of this invention can be used for the topical treatment of psoriasis, eczemas, dermatophytoses, alopecia areata and other dermatological diseases In addition, this invention provides a topical composition which is stable at 37° C. to 45° C. for more than six months thus making it readily dispensable and able to be stored at room temperature in tropical countries.

### DETAILED DESCRIPTION

In accordance with this invention, dithranol is applied topically to the skin in a water and oil nanoemulsion which contains a plurality of bilayer vesicles dispersed therein with the vesicles having a particle size of no greater than 450 nm. These bilayer vesicles are formed with one layer being a lipid phase and the other being an aqueous phase with the lipid phase containing dithranol solubilized in a non-ionic oily liquid lipid material which forms the lipid phase. In accordance with this invention, the composition can contain either dithranol or a mixture of dithranol with salicylic acid solubilized in the lipid phase. In accordance with this invention either dithranol alone or with salicylic acid can form the active ingredient in the topical treatment of dermatological diseases such as psoriasis, etc. In accordance with this invention, the dithranol present in the system in concentrations as high as 1.0% by weight of said composition can be completely solubilized in this composition. In general, these compositions contain dithranol in an amount ranging from about 0.1% to about 1% by weight based upon the weight of the composition. If it is desired to incorporate salicylic acid in the composition, the salicylic acid is present in combination with dithranol in an amount of from about 0.1% to about 3% by weight based upon the weight of the composition and preferably from about 0.5% to about 1.5% by weight based upon the weight of said composition. If salicylic acid is present in combination with dithranol, this combination is entrapped in the lipid phase of the bilayer vesicles within the nanoemulsion.

Solublization is important since Dithranol is insoluble in water and sparingly soluble in hydrocarbon bases, vegetable oils and esters of fatty acids; limiting its concentration below 0.1% in soluble form. It has been very difficult to formulate preparations containing dithranol above 0.1% w/w concentration in soluble form. Surprisingly, it has been found that dithranol and combinations of dithranol and salicylic acid can be solubilized in non-ionic liquid lipid materials such as tocopherol acetate (Vitamin E acetate) which can be used as a vehicle to keep the dithranol in solution in stable form even at high concentrations.

In accordance with the present invention it is discovered that by using a non-ionic oily liquid lipid material which acts as a solubilizer for dithranol, such as α-tocopherol acetate, the dithranol and combinations of dithranol and salicylic acid will be kept in solution entrapped in the vesicles in this oily lipid material which forms the oily phase in both the nanoemulsion and the bilayer vesicles. Any conventional in non-ionic liquid lipid materials, suitable for topical administration, which will solubilize dithranol, can be utilized in formulating the oily lipid phases of this system. Among the preferred non-ionic oily liquid lipid materials are included tocopherol acetate and Arlamol E® (Polyoxypropylene-15-stearyl ether) or mixtures thereof with tocopherol acetate being especially preferred. In formulating the system of this invention, the non-ionic oily liquid lipid material can be generally present in an amount of from about 3% to 40% by weight based upon the weight of the composition, with amounts of from about 5% to 30% by weight based upon the weight of the composition being especially preferred.

The system of this invention achieves enhanced encapsulation of dithranol in solubilized state. Furthermore the composition of the invention surprisingly has been found to be free of undesirable side effects, and provides excellent therapeutic effects in psoriasis, eczemas, dermatophytoses, alopecia areata and other skin disorders when it is administered topically. In clinical tests it has been found that the system of this invention due to the presence of micro-emulsion with mixed vesicles provides effective delivery of dithranol. These advantages are in some part due to—

Presence of nanoemulsion instead of emulsion.

Presence of combination of micro-emulsion with mixed vesicles.

Dithranol entrapment in soluble form.

Preparation mimics liposomal and niosomal delivery system.

It is known that any active ingredient/drug present in solution form diffuses rapidly through the skin as compared to solid dispersed particulate drug. It is an object of the invention to make pharmaceutical compositions containing dithranol in soluble form even at higher drug concentrations so as to make it more effective, where by the active ingredient can penetrate into and through the skin so that the active ingredients are provided continuously and in sufficient quantity at the site of action.

In accordance with this invention, the composition topical administration is in the form of a homogeneous mixture of an oral water nanoemulsion that constitutes an oil lipid phase and an aqueous phase. It can be either an oil in water or water in oil emulsion in which spherical hollow bilayer vesicles like liposomes are present in the single system wherein the dithranol or the dithranol in combination with salicylic acid is present in both the oil globules of the emulsion and in the oil or lipid phase of the bilayer vesicles in a soluble state. Generally, it is preferred that the composition of this invention be a homogeneous mixture of an oil in water nanoemulsion. With the oil lipid phase which is emulsified in the aqueous phase being present in an amount of from 8% to 50% by weight of the composition, preferable from about 5% to about 40% by weight of the composition.

The above mixed vesicular system of this invention which is present as a liquid, if desired, can be thickened to form a gel or cream. This can be done by adding a gelling agent which are thickening agents or viscosity modifying agent to the liquid system. Any conventional gelling agent can be utilized to convert the liquid emulsion of this invention into a cream or gel. Among the preferred gelling agents are included xanthan gum, ethylene oxide, carbopol, hydroxypropyl, methyl or cellulose. The amount of gelling agent to be added to the composition depends upon the ultimate viscosity of the gel or cream that is desired.

In accordance with one embodiment of this invention when salicylic acid is used in combination with dithranol the salicylic acid is also retained in soluble form along dithranol in the mixed vesicular system mimicking niosomes and liposomes. It is also known in the art that pharmaceutical compositions containing dithranol are more effective in combination with salicylic acid and alcoholic extract of coal tar for the treatment of psoriasis. However such preparations are reported have some percentage of alcohol content which may be intensifying the irritation potential to the skin. In addition, if it is desired to further reduce any possible additional irritating effect of alcohol on the skin of the topical composition of this invention one can vacuum dry the alcoholic extract of coal tar and add it to the composition this invention. This vacuum dried coal tar extract is in the form of viscous slurry. Generally in accordance with a preferred embodiment of this invention the coal tar extract is added in an amount of from about 0.1% to about 6.0%, by weight based the weight of the composition with amounts of from about 0.25% to about 2.0% by weight being especially preferred.

It is known in the art that dithranol is very sensitive to oxidative degradation and a variety of known antioxidants such as butylated hydroxyanisole, butylated hydroxytoulene, ascorbic acid, citric acid, oxalic acid, tartaric acid, succinic acid etc. can be present in the preferred compostion of this invention In the present invention, it is preferred to use antioxidants such as BHA and BHT in combination with ascorbic acid to prevent oxidative degradation of dithranol. In one embodiment of the present invention dithranol alone or in combination with salicylic acid are dissolved in a mixture of tocopherol acetate and Arlamol E® (Polyoxypropylene-15-stearyl ether) containing nonionic surfactant and cholesterol. Thus the oily phase formed was homogenized with aqueous phase.

It is known that any active ingredient/drug present in solution form diffuses rapidly through the skin as compared to solid dispersed particulate drug. In accordance with this invention topical pharmaceutical compositions are prepared containing dithranol in soluble form even at higher drug concentrations so as to make it more effective. In tis manner the active ingredients can penetrate into and through the skin so that they are provided continuously and in sufficient quantity at the site of action.

The oil phase may contain lecithin (Phosphotidyl choline) or other synthetic, semisynthetic lecithins alone or in combination such as hydrogenated phosphatidylchloine, Phosphatidylethanolamine, Dipalmatoyl phosphatidylchohne (DPPC), Dipalmatoyl phosphatidylethanohnine (DPPE) Distearoyl phosphatidylcholine (DSPC), Distearoyl phosphatidylethanolamine (DSPE), Dioleylphosphatidylchohne (DOPC), Dioleylphosphatidylethanolamine (DOPE), Phosphatidic acid (PA), Phosphatidylserine (PS), Phosphatidylglycerol (PG) and their hydrogenated analogs.

The preferred carrier for dithranol or dithranol with salicylic acid in accordance with the present invention is tocopherol acetate or mixture of tocopherol acetate. The concentration of tocopherol acetate in the system used for dissolution of dithranol may range from about 5% to about 40% by weight based on the weight of the total composition. The preferred concentration range, however, is from about 3% to about 30%. The concentration of Arlamol E® (Polyoxypropylene-15-stearyl ether) may range from about 1% to about 10% by volume, based upon the volume of the total composition. The preferred concentration range, however, is from about 3% to about 7%.

In the present invention non-ionic surfactant refers to fatty acid esters and ethers of triglycerides, diglycerides or monoglycerides having HLB value in the range of 4 to 16 preferably between 8 to 14. The examples of non-ionic surfactant refers to but not limited to polyoxyl 60 hydrogenated castor oil (Cremophore® RH-60), polyoxyl 35 hydrogenated castor oil (Cremophore® EL), polyoxyl 40 hydrogenated castor oil (Cremophore® RH-40),sorbitan monooleate (Span-80), polyoxyehtylene 20 sorbitan monooleate (Tween-80), Span 20, polyglyceryl-3-oleate (Plurol Olique®) PEG-32 glyceryl stearate (Gelucire53/10).

The concentration of non-ionic surfactant may range from about 0.5% to about 5% by volume based upon the volume of the total composition. The preferred concentration range however is from about 0.75% to about 3% by volume.

The concentration of cholesterol may range from about 0.1 to about 2% by volume of the composition, preferably from about 0.5 to about 1.5%. The lecithin may be present in the mixed vesicular system and the concentration may range from 0.2 to 10% by volume of the total composition, preferably from about 1% to about 6%.

In accordance with the embodiment of this invention other commonly used additives known in the art such as antioxidants, chelating agent may be present. Antioxidants which can be included in accordance with an embodiment of this invention are combinations of butylated hydroxyanisole, butylated hydroxytoulene and ascorbic acid chelating agents includes Disodium EDTA. The concentration of antioxidants and chelating agent may range from about 0.01% to about 0.5% by volume of the total composition, preferably about 0.1% to about 0.5% by volume.

The particle size of the mixed vesicular system obtained by present invention is no greater than 450 nm. Accordingly, another embodiment of the present invention, the mixed vesicular system is preferably gelled. Among the gelling agents which can be used, cellulose derivatives such as hydroxypropyl methyl cellulose, carbopol, xanthan gum and other hydrophilic polymer such as polyethylene oxide. Depending upon the desired viscose, the gelling agent may be generally added in amounts from about 0.1% to about 5% by weight based upon the weight of the composition, preferably 0.5 to about 3% by weight. Concentration of dithranol in the mixed vesicular system according to an embodiment of the present invention may range from 0.01 to about 1.0% by weight in soluble form.

Concentration of salicylic acid in the mixed vesicular system according to an embodiment of the present invention may range from about 0.1% to about 3.0% in soluble form, preferably from about 0.5% to 1.5% by weight. The vacuum dried coal tar extract according to the present invention can be prepared by first dispersing and soaking the coal tar viscous mass in the alcoholic solution of polyoxyethylene 20 sorbitan monolaurate (Tween-80) for 7 days. The extract is then filtered and alcohol is removed form the filtrate by vacuum evaporation at 35° C. temperature in rotary vacuum evaporator to get vacuum dried coal tar extract. Concentration of vacuum dried coal tar extract in the mixed vesicular system according to the present invention may range from about 0.01% to about 6.0% by weight based upon the weight of the composition, preferably from about 0.25% to about 2% by weight.

This invention is directed to the process for preparing an oil and water nanoemulsion with an oil and water phase wherein the nanoemulsion has dispersed therein a plurality of bilayer vesicles retaining dithranol alone or in combination with salicylic acid solubilized in the oily layers of the vesicles. In accordance with this invention, the nanoemulsion is carried out providing a solution of dithranol dissolved in the non-ionic oily liquid lipid material as well as a separate aqueous medium which may contain such excipients as stabilizers, anti-oxidants, etc. dissolved therein. The aqueous medium and the oily solution are mixed together and then homogenized to form a homogeneous emulsion which contains the oily phase and the aqueous phase. Any conventional homogenizer may be used to homogenize this emulsion mixture. This emulsion is next subjected to high pressure homogenization at pressures from about 20,000 psi to about 30,000 psi. In the final step, the homogeneous mixture after being subjected to high pressure homogenization is passed through a 0.45 nm membrane. In this manner, the oil and water nanoemulsion is produced. The oily non-ionic lipid medium such as α-tocopherol acetate prior to mixing with the aqueous phase contains dithranol or dithranol in combination with salicylic acid solubilized in said lipid phase which preferably contains α-tocopherol acetate along with other non-ionic lipids.

This homogenized mixture is then passed through high pressure homogenizer at 20,000 to 30,000 psi pressure followed by passing through 0.45 μm membrane. The resulting system was found to be mixed vesicular system comprising nanoemulsion with vesicles formed by combination of tocopherol acetate, nonionic surfactant, cholesterol and lecithin, encapsulating the dithranol alone or in combination with salicylic acid in soluble form. In the present invention, the oil phase composition is such that after emulsification and high pressure homogenization it forms an oil in water emulsion along with in situ formation of bilayer vesicles composed of the non-ionic oily liquid lipid material such as α-tocopherol acetate and other non-ionic surfactants. In accordance with the method of this invention, the bilayer vesicles are formed in situ after the high pressure homogenization step and are not added separately to the system.

The composition according to the present invention having 0.5% w/w dithranol, 1.15% w/w salicylic acid and 0.58%, w/w vacuum dried coal tar extract were tested clinically on 12 patients with skin psoriasis and found to as equally effective as compared to conventional dithranol ointment containing 1.1% w/w dithranol, 1.15% w/w salicylic acid & 5.3% w/w alcoholic extract of coal tar solution.

### EXAMPLES

The following non-limiting examples set forth herein below illustrate the invention:

Example 1

| | | |
|---|---|---|
| Part A: | | |
| Dithranol- | 0.5% | w/w |
| Salicylic acid- | 1.15 | w/w |
| α-Tocopherol acetate- | 15% | w/w |
| Polyoxypropylene-15-Stearyl ether- | 5% | w/w |
| (Arlamol ®-E) | | |
| Oleic acid- | 0.5% | w/w |
| Butylated hydroxy anisole- | 0.1% | w/w |
| Butylated hydroxy toluene- | 0.1% | w/w |
| Cholesterol- | 0.5% | w/w |
| Polyoxyl 40 hydrogenated castor oil- | 1.0% | w/w |
| Egg lecithin (Lipoid ® E-80 S)- | 3.0% | w/w |
| Ethanol- | 0.75% | w/w |
| Part B: | | |
| Glycerin- | 5.0% | w/w |
| Disodium EDTA- | 0.1% | w/w |
| Ascorbic acid- | 0.5% | w/w |
| Demineralised water- | 51.0% | w/w |
| Part C: | | |
| Xanthan gum- | 2.0% | w/w |
| Methyl paraben- | 0.1% | w/w |
| Propyl paraben- | 0.02% | w/w |
| Sodium hydroxide- | 0.06% | w/w |
| Demineralised water- | 13.0% | w/w |
| Part D: | 0.58% | w/w |
| Coal tar extract (Vacuum drier)- | | |

Heat Part A to 50° C. under nitrogen gas flushing and mix to get homogenous solution. Then cool to room temperature.

Mix Part B with homogenizer.

Add Part A into Part B with homogenization. Then transfer the mixture to high pressure homogenizer (APV Gaulin LAB 40) and homogenize it for 10-12 passes at 30,000 psi pressure. The resulting dispersion is passed through 0.45 μm membrane to obtain the mixed vesicular system with average particle size less than 450 nm.

Part C, xanthan gum gel is prepared separately by hydrating xanthan gum in demineralised water containing parabens at 80-90° C. temperature and allow to cool and soak at room temperature. Then add above homogenized mixed vesicular system and mix to get the gel consistency. Finally add coal tar exact slurry (vacuum dried) and mix thoroughly to get the final composition.

Accelerated Stability Study:

The mixed vesicular composition of Example 1 was filled in collapsible tubes and kept at accelerated temperature conditions, 25° C., 37° C. and 45° C. for 6 months. Samples were periodically analyses for Dithranol and Salicyclic acid content by HPLC analysis. The results are tabulated the following table.

| | Storage | Dithranol content % | Salicylic acid content % |
|---|---|---|---|
| Period | Temperature | by weight | by weight |
| Initial | — | 0.518 | 1.159 |
| 1 Month | 25° C. | 0.521 | 1.159 |
| | 37° C. | 0.518 | 1.158 |
| | 45° C. | 0.517 | 1.156 |
| 3 Month | 25° C. | 0.511 | 1.156 |
| | 37° C. | 0.509 | 1.153 |
| | 45° C. | 0.507 | 1.1516 |
| 6 Month | 25° C. | 0.508 | 1.155 |
| | 37° C. | 0.501 | 1.1514 |
| | 45° C. | 0.494 | 1.148 |

Conclusion The mixed vesicular composition is stable over 6 month even at 37° C. and 45° C. temperature storage as evidenced by it's dithanol content and salicylic acid content not reducing drastically as compared to the initial value.

Example 2

| Part A: | | |
|---|---|---|
| Dithranol- | 0.5% | w/w |
| α-Tocopherol acetate- | 15% | w/w |
| Polyoxypropylene-15 Stearyl ether- | 5% | w/w |
| (Arlamol ®-E) | | |
| Oleic acid- | 0.5% | w/w |
| Butylated hydroxy anisole- | 0.1% | w/w |
| Butylated hydroxy toluene- | 0.1% | w/w |
| Cholesterol- | 0.25% | w/w |
| Polyoxyl 40 hydrogenated castor oil- | 1.0% | w/w |
| Egg lecithin (Lipoid ® E-80 5)- | 3.0% | w/w |
| Ethanol- | 0.75% | w/w |
| Part B: | | |
| Glycerin- | 5.0% | w/w |
| Disodium EDTA- | 0.1% | w/w |
| Ascorbic acid- | 0.5% | w/w |
| Demineralised water- | 51.58% | w/w |
| Part C: | | |
| Xanthan gum- | 2.0% | w/w |
| Methyl paraben- | 0.1% | w/w |
| Propyl paraben- | 0.02% | w/w |
| Sodium hydroxide- | 0.06% | w/w |
| Demineralised water- | 13.0% | w/w |

Heat Part A to 50° C. under nitrogen gas flushing and mix to get homogenous solution. Then cool to room temperature.

Mix Part B with homogenizer.

Add Part A into Part B with homogenization. Then transfer the mixture to high pressure homogenizer (APV Gaulin LAB 40) and homogenize it for 10-12 passes at 30,000 psi pressure. The resulting dispersion is passed through 0.45 μm membrane to obtain the mixed vesicular system with average particle size less than 450 nm.

Part C, xanthan gum gel is prepared separately by hydrating xanthan gum in demineralised water containing parabens at 80-90° C. temperature and allow to cool and soak at room temperature. Then add above homogenized mixed vesicular system and mix to get the gel consistency.

### Example 3

[Therapeutic Composition Without Polyoxypropylne-15-stearyl ether (Arlamol®-E)]

Therapeutic composition similar to that described in Example 1, except α-tocopherol acetate was increased form 15% to 20% by weight and Polyoxypropylne-15-stearyl ether (Arlamol®-E) was omitted.

### Example 4

Therapeutic composition similar to that described in Example 1, except 3.0% w/w Egg lecithin (Lipoid E 80 S) was replaced by Soya lecithin (Lipoid® S75).

### Example 5

Therapeutic composition similar to that described in Example 1, except 3.0% w/w Egg lecithin (Lipoid E 80 S) was replaced by Dipalmitoyl phosphatidylcholine (DPPC).

### Example 6

Therapeutic composition similar to that described in Example 1, except 1.0% w/w polyoxyl-40 hydrogenated castor oil was replaced by 1.0% w/w/polysorbate 80.

### Example 7

Therapeutic composition similar to that described in Example 1, except 1.0% w/w polyoxyl-40 hydrogenated castor oil was replaced by 1.0% w/w span 20 (sarbitan monolaurate).

### Example 8

Therapeutic composition similar to that of Example 1, except 1.0% w/w polyoxyl-40-castor oil was replaced by 1.0% w/w polyglyceryl-3-oleate (Plurol Olique®)

### Example 9

Therapeutic composition similar to that of Example 1, except 0.5% w/w dithranol was substituted by 0.1% w/w dithranol and a-tocopherol acetate reduced from 15% w/w to 10% w/w.

### Example 10

Therapeutic composition similar to that of Example 1, except 0.5% w/w dithranol was substituted by 1.0% w/w dithranol.

### Example 11

(Not forming the part of invention)

Dithranol Ointment

| | | |
|---|---|---|
| Dithranol- | 1.15% | w/w |
| Salicylic acid- | 1.15% | w/w |
| Solution of coal tar (alcoholic)- | 5.3% | w/w |
| Maize starch- | 12% | w/w |

| | |
|---|---|
| White soft paraffin- | q.s. 100% |

Dithranol and maize starch were finely dispersed in melted white soft paraffin at 65-70° C. temperature and cooled to room temperature with mixing. Salicylic acid was dissolved in coal tar solution and added to dithranol paraffin mixture & mixed thoroughly to get ointment consistency.

Clinical Test:

Therapeutic compositions as described in Example 1, 2, and 11 were tested clinically and comparison is being made. Clinical test protocol was followed as described by Van Scott, Eugene J. et al. in U.S. Pat. No. 4287214.

Skin involves in psoriasis is hyperplastic (thickened), erythematous (red or inflamed) and has thick adherent scales. The degree of thickening is such that lesion are elevated up to 1 mm above the surface & adjacent normal skin; erythema is usually an intense red; the thickened adherent scales cause the surface of involved skin to be marked by rough & uneven. These three attributes of thickness, colour & texture as described in U.S. Pat. No. 4287214 were quantified to allow comparative measurement of degree of improvement from topically applied therapeutic compositions—therapeutic composition of the present invention (Example 1 & Example 2) and conventional dithranol ointment (Example 11).

Degree of Improvement

| | None | mid | Moderate | Substantial | Complete |
|---|---|---|---|---|---|
| | (0) | (1+) | (2+) | (3+) | (4+) |
| Thickness | Highly | Detectable | Readily | Barely | Normal |
| | elevated | reduction | apparent | elevated | thickness |
| Texture | Visibly | Palpably | Uneven | Slightly | Visibly and |
| | rough | rough | but not | uneven | palpably |
| | | | rough | | smooth |
| Colour | Intense | Red | Dark Pink | Light pink | Normal skin |
| | Red | | | | colour |

In order to ascertain whether the dithranol compositions of the present invention (Example 1 & Example 2) are therapeutically more efficacious than conventional Dithranol Ointment (Example 11) for topical treatment of psoriasis, a total of more than 12 patients having psoriasis were tested in this study.

The treatment areas in patients having psoriasis were localized lesions 8-20 cm in diameter. The therapeutic compositions were topically applied by the patient in an amount sufficient to cover the treatment. Applications were made two-three times daily and without occlusive dressing. Clinical evaluation of degree of improvement were made of weekly interval. The treatment was continued for 4 weeks unless clearing of disease occurred earlier & evaluation of degree improvement was made. The treatment results are summarized as follows.

TABLE 1
Effect on Psoriasis

Therapeutic
efficiency after

| | Dithranol concentration | 2 weeks | 4 weeks |
|---|---|---|---|
| Example 1 | 0.5% w/w | +3 | +4 |
| Example 2 | 0.5% w/w | +3 | +4 |
| Example 11 | 1.1% w/w | +2 | +3 |

TABLE 2:
Staining and irritation effect of topically applied
composition of skin

| | Dithranol | | | |
|---|---|---|---|---|
| | concentration | | Irritation | Staining |
| Example 1 | 0.5%° | w/w | No irritation | No staining |
| Example 2 | 0.5% | w/w | No irritation | No staining |
| Example 11 | 1.1% | w/w | High irritation | Staining of cloth |
| | | | observed, sometimes | and skin |
| | | | required for the | observed |
| | | | removal from applied | |
| | | | skin | |

Conclusion

Therapeutic compositions of the present invention were found to be nonirritating, non-staining and therapeutically more effective even at low dithranol concentration (0.5% w/w) as compared to conventional Dithranol Ointment containing 1.1% w/w dithranol.

References

U.S. Pat. No. 3,981,996 September 1976 Leigh, et al.

U.S. Pat. No. 5,904,932 May 1976 De Winger; Tom

U.S. Pat. No. 4,954,345 September 1990 Muller; Joseph

U.S. Pat. No. 5,061,486 October 1991 Whitefield; Martin

U.S. Pat. No. 4,367,224 January 1983 Van Scott, et al.

U.S. Pat. No. 5,894,019 April 1999 Hesse, et al.

U.S. Pat. No. 4,895,727 January 1990 Allen; Larry M.

U.S. Pat. No. 4,438,052 March 1984 Weder, et al.

U.S. Pat. No. 6,328,988 December 2001 Uhrich; Kathryn E.

U.S. Pat. No. 4,287,214 September 1981 Van Scott, et al.

U.S. Pat. No. 4,203,969 May 1980 Yarrow, et al.

U.S. Pat. No. 5,358,716 October 1994 Laugier, et al.

GB 2,157,173A October 1985 Josef Muller, et al.

Shyamala, C.; Jayaraman, C.; Topical Delivery of Erythromycin form Various Formulations: An in Vivo Hairless Mouse Study; J. Pharm. Sci.; Vol. 85; N.10; October 1996, 1082-84.

1. A composition comprising an oil and water nanoemulsion having oil and water phases, said nanoemulsion contain a plurality of bilayer vesicles dispersed therein, said vesicles having an average particle size of no greater than 450 nm, one of said bilayers being an oily lipid phase and the other being an aqueous phase, said lipid phase containing dithranol solubilized in a non-ionic oily liquid lipid material which forms said lipid phase.

2. The composition of claim 1, wherein said dithranol is present in the composition in an amount of from about 0.01% to 1% by weight based upon the total weight of said composition.

3. The composition of claim 2, wherein said lipid phase of said vesicle contains salicylic acid together with dithranol solubilized in said lipid phase.

4. The composition of claim 3, wherein salicylic acid is present in the composition in an amount of from about 0.1% to 3% by weight based upon the weight of the composition.

5. The composition of claim 4, wherein the salicylic acid is present in an amount of from about 0.5% to 1.5% by weight.

6. The composition of claim 2, wherein the oil phase of the nanoemulsion and the vesicles contain the same non-ionic oily lipid material.

7. The composition of claim 6, wherein said non-ionic oily lipid is selected from the group consisting of a-tocopherol acetate, polyoxypropylene-15-stearyl ether or mixtures thereof.

8. The composition of claim 7, wherein a-tocopherol acetate comprises said oily phase and is present in an amount of from about 3% to 40% by weight based upon the weight of the composition.

9. The composition of claim 8, wherein said α-tocopherol is present in an amount of from about 5% to 30% by weight based upon the weight of the composition.

10. The composition of claim 9, wherein the oily phase comprises a mixture of polyoxypropylene-15-stearyl ether and α-tocopherol acetate.

11. The composition of claim 2, wherein the composition contains vacuum dried coal tar extract in an amount of from 0.1% to about 6% by weight based upon the weight of the composition.

12. The composition of claim 11, wherein the coal tar extract is present in an amount of from about 0.5% to 1.5% by weight of the composition.

13. The composition of claim 2, having incorporated therein a gelling agent in an amount sufficient to form a gel.

14. The composition of claim 2, having incorporated therein a gelling agent in an amount sufficient to form a cream.

15. The composition of claim 3, wherein the non-ionic oily liquid lipid is polyoxypropylene-15-stearyl ether in an amount of from about 1% to about 40% by weight based upon the weight of the composition.

16. A process for preparing an oil and water nanoemulsion containing an oil phase and a water phase with said nanoemulsion having dispersed therein a plurality of bilayer vesicles which retain dithranol solubilized in the oily phase of the vesicles comprising:
a) providing both a solution of dithranol dissolved in a non-ionic oily liquid lipid material and an aqueous medium;
b) mixing said oily solution with said aqueous medium to form a mixture;
c) homogenizing said mixture to form a homogeneous emulsion containing an oily phase and an aqueous phase;
d) subjecting said homogeneous mixture to high pressure homogenization at pressures of from about 20,000 psi to about 30,000 psi and
e) thereafter passing said high pressure homogenous mixture through a 0.45 nm membrane to produce said oil and water nanoemulsion.

17. The process of claim 16, wherein said dithranol is present in the composition in an amount of from about 0.01% to 1% by weight based upon the total weight of said composition.

18. The process of claim 17, wherein the oil phase of the nanoemulsion and the vesicles are formed from the same non-ionic oily lipid material.

19. The process of claim 18, wherein said non-ionic oily lipid is selected from the group consisting of α-tocopherol acetate, polyoxypropylene-15-stearyl ether or mixtures thereof.

20. The process of claim 19, wherein a-tocopherol acetate comprises said oily phase and is present in an amount of from about 3% to 40% by weight based upon the weight of the composition.

21. The process of claim 20, wherein the aqueous medium contains natural or semi-synthetic lecithins dissolved therein.

22. The process of claim 21, wherein the oily solution contains salicylic acid.
